(19) United States
(12) Patent Application Publication
(10) Pub. No.: US 2026/0175025 A1
(43) Pub. Date: Jun. 25, 2026

(54) VIBRATION THERAPY DEVICE, SYSTEM, AND METHOD

(71) Applicant: Sola Gratia, LLC, Fort Worth, TX (US)

(72) Inventor: Malinda Marie HANSEN, Fort Worth, TX (US)

(73) Assignee: Sola Gratia, LLC, Fort Worth, TX (US)

(21) Appl. No.: 19/155,930

(22) PCT Filed: Feb. 13, 2024
(86) PCT No.: PCT/US2024/015619

§ 371 Date: Aug. 12, 2025

Related U.S. Application Data
(60) Provisional application No. 63/445,234, filed on Feb. 13, 2023.

(51) Int. Cl. A61N 1/36; A61H 23/00

(52) U.S. Cl. CPC A61N 1/36003; A61H 23/00; A61H 2201/165; A61H 2201/5082

(57) ABSTRACT

A wearable vibration therapy device, system and method are described herein for generating a therapeutic vibration from the wearable vibration therapy device. The method includes determining control parameters for controlling the wearable vibration device based on a treatment plan for a target site of a patient's body and providing the control parameters to the wearable vibration therapy device. The method also includes generating an effective frequency and an effective amplitude of the therapeutic vibration via the wearable vibration device, based on the control parameters.

### FIELD OF THE DISCLOSURE

The present disclosure relates to a vibration therapy apparatus, a system associated with the vibration therapy apparatus, and associated methodology for using the system.

### BACKGROUND

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

### SUMMARY

According to aspects of the disclosed subject matter, a vibration therapy system includes a vibration device and a controller configured to control vibration frequencies, amplitudes, durations of therapy, and number of treatments for the vibration device. In some aspects of the disclosed subject matter, a variable vibration frequency, amplitude, and duration of the vibration therapy may be adjusted accordingly to address neuromodulation response during rehabilitation for issues including, but not limited to, injury recovery, fracture healing, post-surgical rehabilitation, sore muscles, muscle spasms, and/or blood flow. In some embodiments, the vibration therapy system can deliver a calming or stimulating massage to the treated area. Variations in the frequencies, amplitudes and durations of the vibration therapy can aid in stimulating the parasympathetic nervous system and the relaxation of the muscles. Vibration therapy with increased frequencies and amplitudes can be utilized to increase blood flow and augment natural repatterning as well as increase hyaluronic acid of muscle tone. As such, reduced pain, rapid recovery for healing tissue, improved muscle strength, and/or circulation during rehabilitation are several benefits and outcomes that may be achieved in the areas of musculoskeletal rehabilitation and neural/motor patterning. Vibration therapy has been used to treat various ailments, including improving gait parameters in elderly patients, improve quadricep function post-surgically, decrease spasticity in post-stroke populations, ligament sprains, muscle strains, tendonitis, joint inflammation, plantar fasciitis, metatarsalgia, facet irritation, impingement syndrome, bursitis, rheumatoid arthritis, osteoarthritis and scar tissue adhesion.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 illustrates a vibration therapy system, according to one or more aspects of the disclosed subject matter;

FIG. 2 illustrates the wearable vibration device according to some aspects of the disclosed subject matter;

FIG. 3 is a cross-sectional view of the wearable vibration device according to some aspects of the disclosed subject matter;

FIG. 4 illustrates an adjustable strap according to some aspects of the disclosed subject matter;

FIG. 5 illustrates a second wearable vibration device according to some aspects of the disclosed subject matter.

FIG. 6 illustrates a cross-sectional view of the second wearable vibration device according to some aspects of the disclosed subject matter.

FIG. 7 is a hardware block diagram of the user device according to one or more exemplary aspects of the disclosed subject matter.

### DETAILED DESCRIPTION

The description set forth below in connection with the appended drawings is intended as a description of various embodiments of the disclosed subject matter and is not necessarily intended to represent the only embodiment(s). In certain instances, the description includes specific details for the purpose of providing an understanding of the disclosed subject matter.

However, it will be apparent to those skilled in the art that embodiments may be practiced without these specific details. In some instances, well-known structures and components may be shown in block diagram form in order to avoid obscuring the concepts of the disclosed subject matter.

Reference throughout the specification to “one embodiment” or “an embodiment” means that a particular feature, structure, characteristic, operation, or function described in connection with an embodiment is included in at least one embodiment of the disclosed subject matter. Thus, any appearance of the phrases “in one embodiment” or “in an embodiment” in the specification is not necessarily referring to the same embodiment. Further, the particular features, structures, characteristics, operations, or functions may be combined in any suitable manner in one or more embodiments. Further, it is intended that embodiments of the disclosed subject matter can and do cover modifications and variations of the described embodiments.

It must be noted that, as used in the specification and the appended claims, the singular forms “a,” “an,” and “the” include plural referents unless the context clearly dictates otherwise. That is, unless clearly specified otherwise, as used herein the words “a” and “an” and the like carry the meaning of one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein, merely describe points of reference and do not necessarily limit embodiments of the disclosed subject matter to any particular orientation or configuration. Furthermore, terms such as “first,” “second,” “third,” etc., merely identify one of a number of portions, components, points of reference, operations and/or functions as described herein, and likewise do not necessarily limit embodiments of the disclosed subject matter to any particular configuration or orientation.

Referring now to the drawings, wherein like reference numerals designate identical or corresponding parts throughout the several views. The drawings are representative of one or more aspects of the disclosed subject matter; however, the drawings may not be drawn to scale.

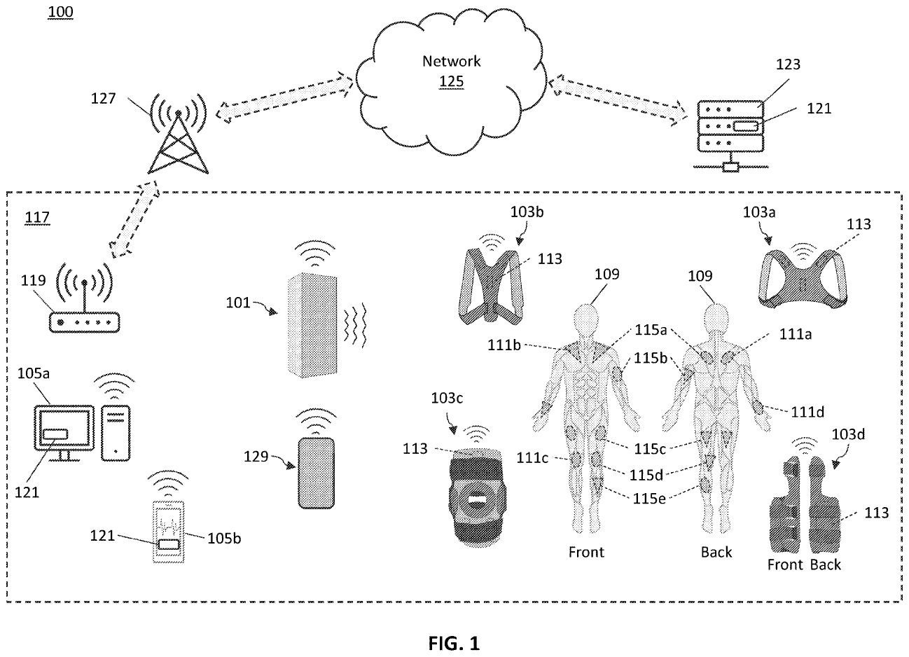

FIG. 1 illustrates a vibration therapy system 100 according to one or more aspects of the disclosed subject matter. The vibration therapy system 100 comprises at least one wearable vibration device 101, an adjustable strap 103, a user device 105, and a vibration technology application 121, in accordance with some embodiments. The vibration technology application 121 may also be referred to herein as a cloud-based vibration technology system. The vibration technology application 121 can be configured to control the operation of the wearable vibration device 101. For example, the vibration technology application 121 can control activation, deactivation, and/or set control parameters for the wearable vibration device 101. According to some embodiments, the control parameters of the wearable vibration device IOI include but are not limited to: a timer for the duration of vibration therapy, amplitude of vibration, vibration frequency, detect/identify target site, and/or date and time stamp for recorded activity. According to one or more aspects of the disclosed subject matter, the vibration frequency may be set to an inclusive range between 40 Hz and 120 Hz. In another embodiment, the vibration frequency may be set to an inclusive range of 20 HZ and 150 Hz. However, any suitable range may be used for the vibration frequency. According to one or more aspects of the disclosed subject matter, the vibration amplitude may be set to an inclusive range between 0.1 mm and 3.0 mm. However, any suitable range may be used for the amplitude of vibration.

The at least one wearable vibration device 101 may be configured to attach to the adjustable strap 103 and contacts a target site 111 of a patient's body 109 when the adjustable strap 103 is worn by the patient. Examples of attachment location(s) 113 are indicated by the dashed lines on each of the adjustable strap(s) 103 illustrated in FIG. 1. However, any suitable attachment area may be used to place the wearable vibration device 101.

According to some aspects of the disclosed subject matter, the at least one wearable vibration device 101 may be configured to attach to the adjustable strap 103a (e.g., a clavicle brace) and contact target site 111a (e.g., the retractor muscles of the scapula). As another example, the at least one wearable vibration device 101 may be attached to the adjustable strap 103b (e.g., suspender style brace) and contact target site 111b (e.g., the upper trapezius muscle, posterior shoulder). In still another example, the at least one wearable vibration device 101 may be attached to the adjustable strap 103c (e.g., knee brace) and contact target site 111c (e.g., the rectus femoris muscle). In yet another example, the at least one wearable vibration device 101 may be attached to the adjustable strap 103d (e.g., wrist brace) and contact target site 111d (e.g., the flexor carpiulnaris muscle). The adjustable straps 103a, 103b, 103c and 103d and the target sites 111a, 111b, 111c, and 111d may be collectively or individually referred to herein as the adjustable strap(s) 103 and the target area(s) 111, respectively. Although examples are illustrated and described herein with regard to the adjustable straps 103a, 103b, 103c and 103d and the target sites 111a, 111b, 111e, and 111d, the wearable vibration device 101 may be customized to attach to any other adjustable strap(s) 103 and contact any other target site(s) 111 of the patient's body 109 without departing from the spirit and scope of the present invention.

In some embodiments, the placement of the at least one wearable vibration device 101 may be in a location other than a location of an injured muscle, an ailing tendon, or a soft tissue injury. In such cases, an antagonistic muscle pairing may be subject to the vibration therapy in order for the injured muscle or ailing tendon to undergo physical therapy without the added strain of the antagonistic muscle working against the muscle, tendon, or soft tissue performing the physical therapy. Several examples of antagonistic pairing muscles 115 are illustrated with target muscles indicated with dashed ovals and antagonistic pairing muscles (e.g., 115a, 115b, 115c, 115d, 11Se, or the like) being indicated with dashed triangles in FIG. 1. Muscles to be targeted can include any antagonistic pairing anywhere on the patient's body 109. Examples of antagonistic pairing muscles include but are not limited to: scapular retractors/pectoralis/anterior deltoid; latissimus/pectoralis; bicep/triceps; elbow extensors/flexors; gluteus muscles/hip flexor/quadriceps/adductors/paraspinal muscles; quadriceps/hamstrings; gastrocnemius/anterior tibialis. For example, to relieve low back pain, the at least one wearable vibration device 101 may be placed on the gluteal musculature rather than the paraspinals of the low back. In doing so, the neuromuscular and physiologic response of the muscle spindles activate the gluteal muscles which, in turn, reciprocally inhibit the tension placed on the paraspinal musculature. In another example, to aid in the recovery of a hamstring strain, the at least one wearable vibration device 101 would be placed on the quadriceps tendon to reciprocally inhibit the hamstring, thus taking undue tension off the hamstrings.

According to some aspects of the disclosed subject matter, other therapies can be provided with/or in addition to the vibration therapy. In some embodiments, the at least one wearable vibration device 101 may further comprise an optional heating element for applying a suitable degree of heat to the target site(s) 111 and/or the antagonistic pairing muscles of the patient's body 109. The additional heat can provide increased blood flow and/or flexibility to the applied muscles.

In some embodiments, the wearable vibration device 101 is configured to wirelessly communicate to the user device 105, for example, over a local area network 117 via the wireless access point 119. In such embodiments, the user device(s) 105 may be configured to control the wearable vibration device 101 through the vibration technology application 121 installed on the user device(s) 105.

According to one or more aspects of the disclosed subject matter, the wearable vibration device 101 is portable and can be attached to any suitable adjustable strap 103 (e.g., harness), anchored to clothing, and/or can be fixed to a flexible fabric adhesive patch (e.g., non-woven polyester) for applying vibration therapy to any suitable target site(s) 111 of the patient's body 109. As such, there is no movement or displacement of the wearable vibration device 101 during the vibration therapy and/or during movement of the patient's body 109 during physical therapy. According to some embodiments, the adjustable strap 103 may be made of a flexible neoprene, non-woven polyester (or comparable) fabric with a velcro, buckle, strap or other attachments for ease of variable sizing and fitting the patient's body 109. Although examples are disclosed herein with regard to the wearable vibration device 101 being attached to an adjustable strap 103, the wearable vibration device 101 may also be configured to be held in place by the user (e.g., a certified healthcare provider, a physical therapist, a medical provider, the patient, an assistant, or the like) during the vibration therapy and/or during movement of the patient's body 109 during physical therapy without departing from the spirit and scope of the disclosed subject matter.

According to some embodiments, the wearable vibration devices 101 may be configured to wirelessly communicate to one or more of the user devices 105. In some aspects of the disclosed subject matter, the wireless access point 119 provides communications between the user device 105 and/or to a network server 123 over a wide area network 125 (e.g., the internet, a campus area network, a city area network, or the like). In some embodiments, the wireless access point 119 may communicate between one or more of the user device 105 and/or the wearable vibration devices 101 and the network server 123 via a radio access network 127 (e.g., UMTS, 5G, or the like) interfacing the wide area network 125. However, the wireless access point 119 may also be configured to communicate to the user device 105 and/or the network server 123 via a wired and/or fiber optic network.

The user device 105 may be any user computing device (e.g., a computer (PC), laptop, smartphone, cellular phone, notebook computer, tablet, netbook, a personal digital assistant (PDA), or any other programmable electronic device capable of communicating with other devices via the local area network 117 and via the wide area network 125. The user device 105 can be operated by a user to monitor and interact with the wearable vibration device 101 and/or the network server 123 as further described herein. For example, a user (e.g., qualified healthcare provider) can use the user device 105 to select their preferred settings and modes of operation of the wearable vibration device 101 in the vibration therapy system 100, as further described herein. In one embodiment, the user device 105 interacts with the vibration therapy system 100 via the vibration technology application 121 (e.g., a mobile application, web application, etc.) as further described herein.

In some embodiments, a first user device 105a may display data obtained from wearable vibration devices 101 in a first format, for example a web browser, that is suitable to a desktop computer. The same or similar data may be displayed on a second user device 105b (e.g., smartphone) in a second format, for example a compressed cell phone video format (e.g., 3GPP, MPEG-4, RTSP, or the like). In some embodiments, the information displayed on the first user device 105a may be segmented into separate displays and formatted appropriately for display on the second user device 105b. The first user device 105a and the second user device 105b may be collectively referred to herein as the user device(s) 105.

The vibration technology application 121 may be a software application and/or a hardware application residing in whole or in part on the network server 123, according to one or more aspects of the disclosed subject matter. In some of the embodiments, part or all of the software application and/or the hardware application resides in the user device 105 and cooperates with the other parts of the software application and/or hardware application residing in the network server 123 over the wide area network 125. The software application and/or a hardware application may reside on the first user device 105a (e.g., desktop computer) and/or the second user device 105b (e.g., smartphone).

According to one or more aspects of the disclosed subject matter, the wearable vibration devices 101 may be used in different clinical scenarios for vibrometry. The following listing of examples is non-exhaustive and any suitable control parameters of vibration therapy may be used for the treatment of still other example ailments based on the target site 111 of the patient's body 109 without departing from the spirit and scope of the disclosed subject matter.

In an example scenario, one or more wearable vibration devices 101 can be used with the adjustable strap 103b to provide vibration therapy at a target site 111 (e.g., gluteal musculature, paraspinals, and/or quadriceps/hamstrings) to treat mechanical low back pain. In such embodiments, the control parameters can be set, for example, to an amplitude in an inclusive range between 0.5 mm-3.0 mm, a vibration frequency in an inclusive range between 40 Hz-120 Hz with an average range between 80 Hz-120 Hz, and a time in an inclusive range between 5-15 minutes, up to 60 minutes 2-3×/week or 15 minutes once daily.

In another example scenario, one or more wearable vibration devices 101 can be used with the adjustable strap 103c to provide vibration therapy at a target site 111 (e.g., proximal quadriceps tendon) to treat osteoarthritis of the knees. In such embodiments, the control parameters can be set, for example, to an amplitude in an inclusive range between 0.5 mm-3.0 mm, a vibration frequency in an inclusive range between 80 Hz-120 Hz, and a time in an inclusive range between 15-30 minutes, up to 60 minutes 2-3×/week or 20 minutes once daily.

In still another example scenario, one or more wearable vibration devices 101 can be used with the adjustable strap 103a to provide vibration therapy at a target site 111 (e.g., retractor musculature mid back, lower trapezius) to treat upper crossed syndrome. In such embodiments, the control parameters can be set, for example, to an amplitude in an inclusive range between 0.5 mm-1.5 mm, a vibration frequency in an inclusive range between 60 Hz-120 Hz, and a time in an inclusive range between 5-15 minutes, up to 60 minutes 2-3×/week or 10-15 minutes once daily.

In yet another example scenario, one or more wearable vibration devices 101 can be used with the adjustable strap 103c to provide vibration therapy at a target site 111 (e.g., gluteal musculature (hip), proximal quadriceps tendon (knee)) to treat post-surgical total hip/total knee. In such embodiments, the control parameters can be set, for example, to an amplitude in an inclusive range between 0.5 mm-3.0 mm, a vibration frequency in an inclusive range between 80 Hz-120 Hz, and a time in an inclusive range between 15-30 minutes, up to 60 minutes 2-3×/week or 20 minutes once daily.

In still yet another example scenario, one or more wearable vibration devices 101 can be used with the adjustable strap 103c to provide vibration therapy at a target site 111 (e.g., distal anterior tibialis tendon, proximal gastrocnemius tendon, distal Achilles tendon) to treat acute ankle sprain. In such embodiments, the control parameters can be set, for example, to an amplitude in an inclusive range between 0.1 mm-0.5 mm, a vibration frequency in an inclusive range between 60 Hz-120 Hz, and a time in an inclusive range between 5-15 minutes, up to 60 minutes 3×/week or 10 minutes once daily. In another embodiment, control parameters can be set to a vibration frequency in an inclusive range between 40 Hz-120 Hz, for 30 minutes once daily

In the forgoing examples, control parameters of vibration therapy are provided for the treatment of certain ailments based on the target site 111 of the patient's body 109, the clinical scenario, and other recommendations of a certified healthcare provider. In one or more aspects of the disclosed subject matter, the vibration technology application 121 can determine and recommend a vibration treatment plan (e.g., programmed clinical protocols) based on a clinical scenario set by the user (e.g., a certified healthcare provider, a physical therapist, a medical provider, or the like). In some embodiments, the appropriate target site(s) 111 can be determined based on the vibration treatment plan and/or clinical scenario set by the user. In such embodiments, the one or more wearable vibration devices 101 and/or the vibration technology application 121 may provide the user the treatment plans and positioning information for each of the one or more wearable vibration devices 101. For example, the user (e.g., a certified healthcare provider) may perform muscle tests on the patient and/or may provide input from the patient's medical records (e.g., ultrasound scans, cat scans, X-rays, or the like) to provide details and/or make recommendations for a patient's vibration treatment for the one or more wearable vibration devices 101. In such embodiments, the details provided and/or recommendations made can be received from the network server 123 over the wide area network 125 and a vibration treatment determined by the vibration technology application 121 can be wirelessly transmitted to the one or more wearable vibration devices 101 or may be entered directly by the user via a user interface of the one or more wearable vibration devices 101.

In some embodiments, when more than one wearable vibration device 101 is to be used for a treatment plan, each of the wearable vibration devices 101 may be uniquely programmed or set by the user according to the treatment plan. In some embodiments, the wearable vibration devices 101 are programmed by commands received from the vibration technology application 121. Such commands can be received by the wearable vibration devices 101 wirelessly (e.g., Wi-Fi, Bluetooth, or the like) and/or via a wired connection (e.g., USB).

In one or more aspects of the disclosed subject matter, the vibration technology application 121 can validate a placement of the wearable vibration device 101 at an appropriate target site 111 of the patient's body 109 based on a clinical scenario set by the user (e.g., a certified healthcare provider, a physical therapist, a medical provider, or the like). In such embodiments during placement, the one or more wearable vibration devices 101 and/or the vibration technology application 121 may provide the user an indication that positioning of the wearable vibration device 101 at the appropriate target site 111 has been obtained. This may be helpful when the user placing the wearable vibration device 101 is not the user that reviewed and/or set the treatment plan. For treatment plans using more than one wearable vibration device 101, this is helpful to prevent placing a first wearable vibration device 101 having been set or programmed with a first unique treatment plan at a target site 111 intended for a second wearable vibration device 101 having been set or programmed with a second unique treatment plan different from the first. In some embodiments, the indication is an audible indication (e.g., beep, verbal cue, ring, chirp, or the like); a vibration (e.g., haptic feedback, or the like); and/or a visual indication (e.g., light, led, text, icon display, or the like).

In such embodiments, the user device 105 and/or the wearable vibration device 101 may be equipped with technology to determine a position of the wearable vibration device 101 in relation to a target area 111 of the patient's body 109. For example, a video technology (e.g., camera and object identification software), an ultrasound technology (e.g., ultrasound transceiver), near field communication technology (e.g., NFC transponder implanted in a knee or hip replacement structure, or a subcutaneous microchip implanted during surgery), and/or positional sensors (accelerometers, gyroscopes, or the like) may be used to determine the correct placement and orientation of wearable vibration device 101.

In some embodiments the vibration technology application 121, when executed by circuitry of the network server 123 and/or circuitry of the user device 105, causes the circuitry to generate reports (e.g., usage data) related to the wearable vibration devices 101 and display the reports on the user device 105. An example report may include a patient's identity, identifier(s) of the one or more wearable vibration devices 101, dates and times of use, frequency of use, target site(s) 111 of the patient's body 109, settings used (e.g., amplitudes, frequencies (e.g., Hz)), duration of use, progress of physical therapy, daily feedback survey results (e.g., patient questionnaire), or the like. In some embodiments, the report may include charts of the patient's progress of rehabilitation correlated with the vibration therapy over time.

According to some embodiments, an optional biomarker device 129 may be used in connection with the at least one or more wearable vibration devices 101. The optional biomarker devices 129 may be a wired device or a wireless device and is equipped to transmit measurements to the at least one or more wearable vibration devices 101 and/or the user device 105. Based on these measurements, determinations of the for the degree of repair for an injury, the amount of therapy completed, and/or an increased performance level for the target site 111 of the patient's body 109. Examples of optional biomarker devices 129 include but are not limited to: an EMG device, an EKG device, an oxygen sensor, a thermometer, or the like. In the case of the optional biomarker device 129 being an EMG device, measurements of muscle contractions and/or measures of strength may be used to assess the progress of the physical therapy. In some aspects of the described subject matter, the at least one wearable vibration device 101 and the optional biomarker device 129 (e.g., EMG device) are applied to an antagonistic pairing muscle of the patient's body 109. In such embodiments, the at least one wearable vibration device 101 may use the measurements obtained from the optional biomarker device 129 to indicate a degree of therapy completed and/or to indicate that an antagonistic pairing muscle is ready for physical therapy to begin.

The network server 123 can represent one or more servers communicably coupled to the wearable vibration devices 101 and/or the user device 105 via the wide area network 125. The network server 123 can be configured to perform various processing for the vibration therapy system 100 as further described herein. Additionally, the network server 123 can represent a dedicated bank of servers, cloud-based processing, and/or a serverless computing system corresponding to a virtualized set of hardware resources.

The wide area network 125 can be a public network, such as the Internet, or a private network, such as an LAN or WAN network, or any combination thereof and can also include PSTN or ISDN sub-networks. The wide area network 125 can also be wired, such as an Ethernet network, or can be wireless such as a cellular network including EDGE, 3G, 4G, and 5G wireless cellular systems. The wireless network can also be Wi-Fi, Bluetooth, or any other wireless form of communication that is known.

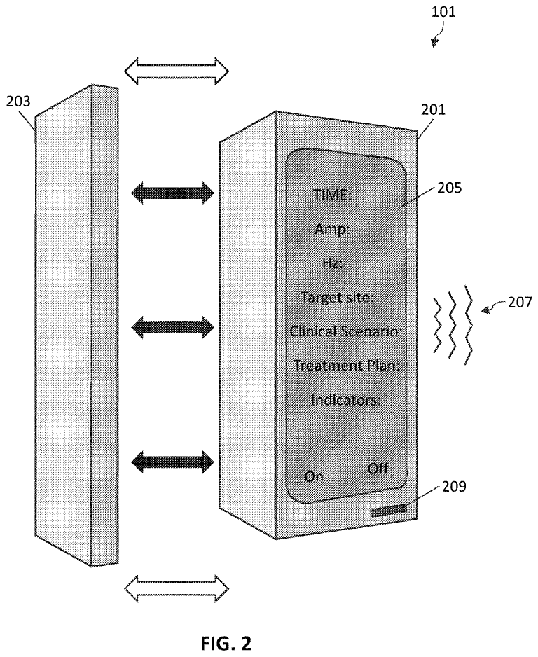

FIG. 2 illustrates the wearable vibration device 101 according to some aspects of the disclosed subject matter. The wearable vibration device 101 comprises a vibration element 201 and an attachment plate 203. In accordance with some embodiments, the attachment plate 203 is magnetically attracted to the vibration element 201 and is able to hold the vibration element 201 to an adjustable strap 103 positioned therebetween. The attachment plate 203 may be magnetic and the vibration element 201 may comprise a metal base or metal housing that is attracted to the attachment plate 203, in some embodiments. In other embodiments, the vibration element 201 may be magnetic and the attachment plate 203 may comprise a metal surface that is attracted to the vibration element 201. However, in still other embodiments, the vibration element 201 and the attachment plate 203 comprise magnets and/or maybe magnetic which are attracted to one another.

According to one or more aspects of the described subject matter, the wearable vibration device 101 may comprise a user interface 205 (e.g., control buttons, switches, touch screen, or the like) to manually set the control parameters to be used during the vibration therapy. For example, the user interface 205 illustrated in FIG. 2 is a touchscreen that allows a user to input the control parameters (e.g., time, amplitude, frequency, target site, clinical scenario, treatment plan, or the like). The user interface 205 may also comprise control buttons to turn the wearable vibration device 101 on and off The user interface 205 may further display indicators, as discussed herein, to the user. However, these controls and control parameters may also be entered via the user device(s) 105.

When placed at the target site(s) 111 of the patient's body 109 and activated, the wearable vibration device 101 performs the vibration therapy and produces vibrations 207 (e.g., therapeutic vibrations, therapeutic waveforms, therapeutic reverberation, therapeutic resonance, therapeutic percussion, or the like) according to the set control parameters. The downward force of the magnetic attraction between the attachment plate 203 and the vibration element 201 provides the force necessary to provide an effective amplitude.

The vibration element 201 comprises a connection port 209 according to one or more aspects of the disclosed subject matter. The connection port 209 may be used to charge an internal battery of the wearable vibration device 101. The connection port 209 may also be used to transfer data to and/or from the wearable vibration device 101. In some other embodiments, the wearable vibration device 101 may comprise an induction coil used to wirelessly charge the internal battery. In such embodiments, the wearable vibration device 101 may not have the connection port 209.

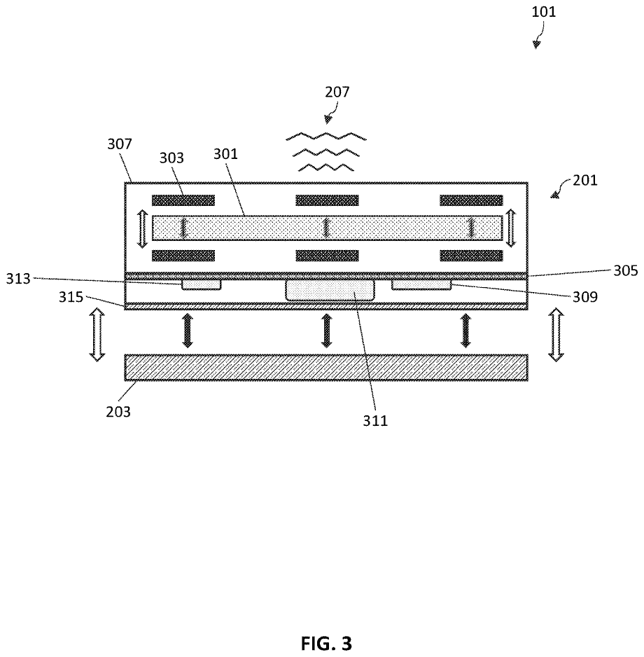

FIG. 3 is a cross-sectional view of the wearable vibration device 101 according to some embodiments. The vibration element 201 comprises a vibration mass 301, magnetic actuators 303, and a control unit 305 (e.g., control board, printed circuit board (PCB)) enclosed within a housing 307 of the vibration element 201. As indicated by the small white arrows, the vibration mass 301 during operation is shifted between the upper magnetic actuators 303a and the lower magnetic actuators 303b according to one or more aspects of the disclosed subject matter. The shifting of the vibration mass 301 is accomplished via alternating between a magnetic field activated at the upper magnetic actuators 303a and a magnetic field activated at the lower magnetic actuators 303b thereby producing the vibrations 207 in a vertical direction and according to the set control parameters (e.g., amplitude, frequency (Hz)). However, other mechanisms (e.g., vibration generators, vibration motors, rotating mass motors, linear resonant actuators (LRA), solenoid actuators, piezoelectric actuator, wave generator, shakers, vibration coils, haptic vibration systems, or the like) may be used to create the vibrations 207 and the vibrations 207 may be amplified in an suitable direction (e.g., longitudinal direction, latitudinal direction, radial direction, or the like). In some embodiments, the vibrations 207 are produced using an eccentric rotating mass (ERM) vibrating motor or a responsive linear resonant actuator (LRA) motor may be used. The vibration mass 301 may be a substantially round, circular, oval, square, prism or rectangular. In one embodiment, the vibrational mass 301 is comprised of at least two vibrational masses that are physically connected in series. In one embodiment, a thermally conductive foam material is placed on the vibrational element 201 that heats the vibration element 201 to a maximum temperature of 40° C. (104° F.). Adding a thermally conductive foam on the vibrational element 201 delivers heat safely to the skin without risks of skin burns. In one embodiment, the thermally conductive foam is positioned on the vibrational mass 301.

The control unit 305 controls the activation of the upper magnetic actuators 303a and the lower magnetic actuators 303b and thus the shifting of the vibration mass 301. In some embodiments, the control unit 305 comprises a processor 309 (e.g., a semiconductor processor, system on chip, integrated circuit, or the like), a battery 311 (e.g., rechargeable lithium battery), and one or more integrated circuits 313 (e.g., wireless transceiver, random access memory, or the like). The processor 309 is configured to control the functional operations of the wearable vibration device 101 in response to the instructions of the vibration technology application 121 and according to the control parameters set for the vibration therapy. According to some embodiments, the vibration element 201 comprises a mounting surface 315 on a side of the vibration element 201 that opposes the attachment plate 203. In some embodiments, the mounting surface 315 is attached to an outer surface of the vibration element 201. However, the mounting surface 315 may be integrated into the outer surface of the vibration element 201 or may be enclosed within the vibration element 201. In some embodiments, the mounting surface 315 and attachment plate 203 are magnetically attracted to one another as indicated by the large black arrows. The attachment plate 203 can be releasably attached to the mounting surface 315, as indicated by the large white arrows, making for easy positioning of the wearable vibration device 101 on an adjustable strap 103 disposed therebetween.

Although examples of attachment of the wearable vibration device 101 are disclosed with respect to a magnetic/mechanical attachment to the adjustable strap 103, any suitable means of attachment may be used. For example, one or more of the vibration element 201 and/or attachment plate(s) 203 may be sewn into the clothing and/or adjustable strap(s) 103 at one or more desirable attachment location(s) 113 without departing from the spirit and scope of the disclosed subject matter. In other embodiments, fasteners may be used to attach the wearable vibration device 101 to the adjustable strap(s) 103. Fasteners include but are not limited to Velcro™, snaps, hooks, zippers, or the like. In yet other embodiments, the wearable vibration device 101 may be adhered directly to the patient, for example using a flexible fabric adhesive patch (e.g., non-woven polyester). In all cases, the wearable vibration device 101 remains in the appropriate position of the target site 111 of the patient's body 109 during the vibration therapy.

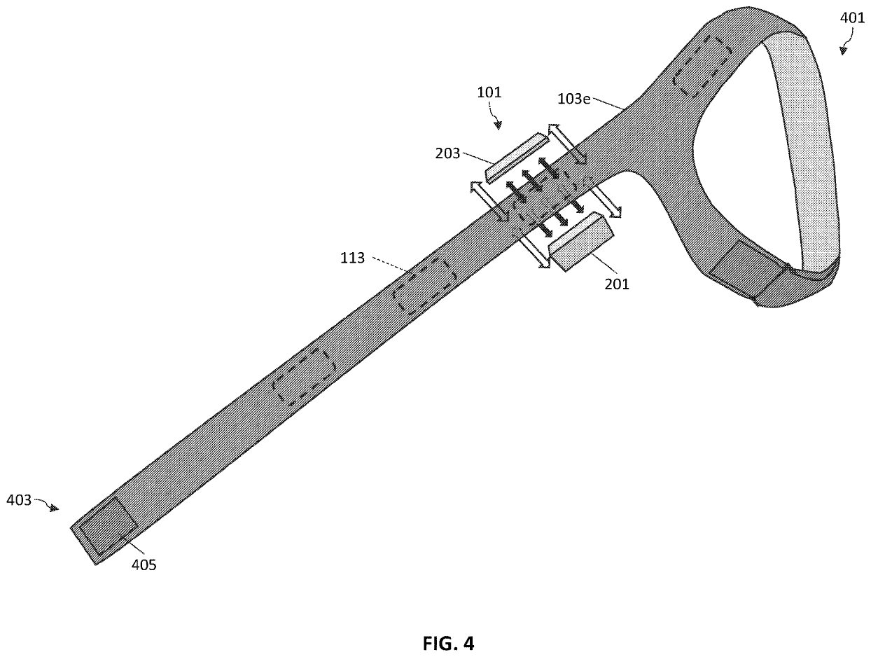

FIG. 4 illustrates the adjustable strap 103e according to another embodiment. In particular, the adjustable strap 103e comprises a loop end 401 and a straight end 403. The loop end 401 may be adjustable and may be worn by the patient around the shoulder, the neck, the leg, the arm with the straight end 403 allowed freedom to wrap around a portion of the patient's body 109. The straight end 403 may comprise a fastener 405 (e.g., velco1M, or the like) to secure the straight end 403 to the adjustable strap 103e itself or to another adjustable strap or article of clothing.

FIG. 4 further illustrates the attachment of the vibration element 201 and attachment plate 203 with the adjustable strap 103e disposed therebetween. The small black arrows show the magnetic attraction between the attachment plate 203 arranged above the adjustable strap 103e and the vibration element 201 arranged below the adjustable strap 103e. The larger white arrows indicate the movement of the attachment plate 203 and the vibration element 201 towards one another ultimately anchoring one to another with the adjustable strap 103e sandwiched therebetween. In one embodiment, heating elements 400 are positioned along the length of the adjustable strap 103e to heat the user's body while the device is in use. In another embodiment, a heating element is incorporated into the vibration device to deliver heat.

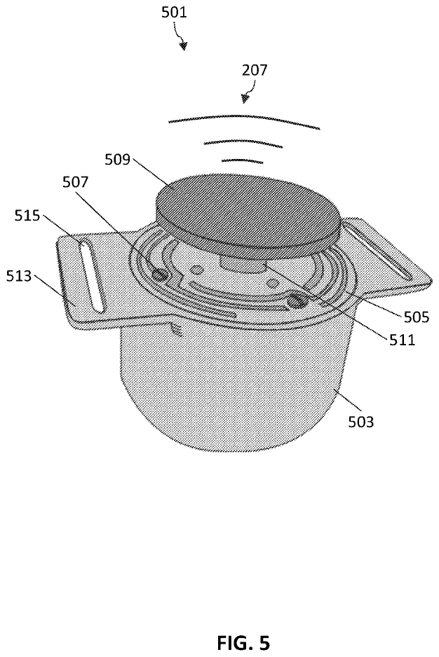

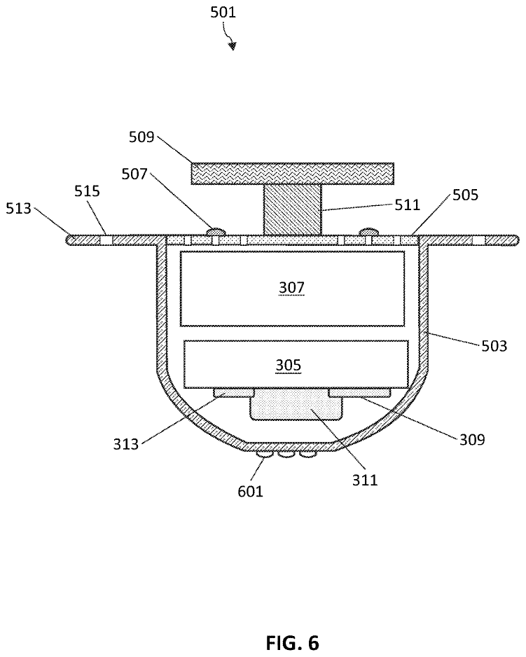

FIGS. 5 and 6 illustrate a second wearable vibration device 501 according to another embodiment. In particular, the second wearable vibration device 501 comprises a housing base 503, a housing lid 505, hardware fasteners 507, a paddle 509, and a stem 511. The housing base 503 comprises slotted flanges 513 having slots 515, according to some embodiments. The slots 515 accommodate the adjustable strap(s) 103 and/or may be anchored to the adjustable strap(s) 103 via a suitable mounting means (e.g., clips, holders, ties, velcro1M, or the like), in accordance with one or more aspects of the disclosed subject matter. The housing lid 505 is attached to the housing base 503 via hardware fasteners 507 (e.g., screws). The paddle 509 is attached to the stem 511 which extends through the housing lid 505. According to some embodiments, the paddle 509 is detachable from the stem 511 and paddles 509 of different sizes and shapes may be attached depending on the vibration treatment being administered. In some embodiments, the paddles 509 may be constructed of certain materials (e.g., soft foam, rubber, metal, plastic, combinations thereof, or the like) and have different shapes (e.g., flat disc, round, arch, or the like) or different length shafts (e.g., 1 cm, 5 cm, or the like). During operation, the second wearable vibration device 501 produces the vibrations 207 according to the set control parameters (e.g., amplitude and frequency (Hz)).

FIG. 6 illustrates a cross-sectional view of the second wearable vibration device 501 according to some aspects of the disclosed subject matter. FIG. 6 further illustrates buttons 601 at the bottom of the housing base 503, according to some embodiments. The buttons 601 may be used to activate and deactivate the second wearable vibration device 501 and set the control parameters for the vibration therapy. However, the control parameters can be set via the application 121 on one of the user devices 105.

According to some aspects of the disclosed subject matter, the buttons 601 activate vibration therapy associated with a desired outcome (e.g., improved blood flow, relieve muscle spasm, aid in rehabilitation, or the like). For example, one of the buttons 601 may be designated to activate a vibration therapy suitable for improving blood flow at the target site 111 of the patient's body 109. As another example, one of the buttons 601 may be designated to activate a vibration therapy suitable for relieving muscle spasm at the target site 111 of the patient's body 109. In still another example, one of the buttons 601 may be designated to activate a vibration therapy suitable for aiding in rehabilitation at the target site 111 of the patient's body 109. In some embodiments, one or more of these buttons may be used in combinations to provide any combination of vibration therapy suitable for treatment at the target site 111 of the patient's body 109. In some embodiments, the vibration therapy for two or more of the buttons 601 may be activated during treatment to allow for multiple treatments to be performed during the vibration therapy. In some embodiments, the multiple treatments may be combined as a series of treatments or may be combined as a pattern of treatments (e.g., alternating treatments).

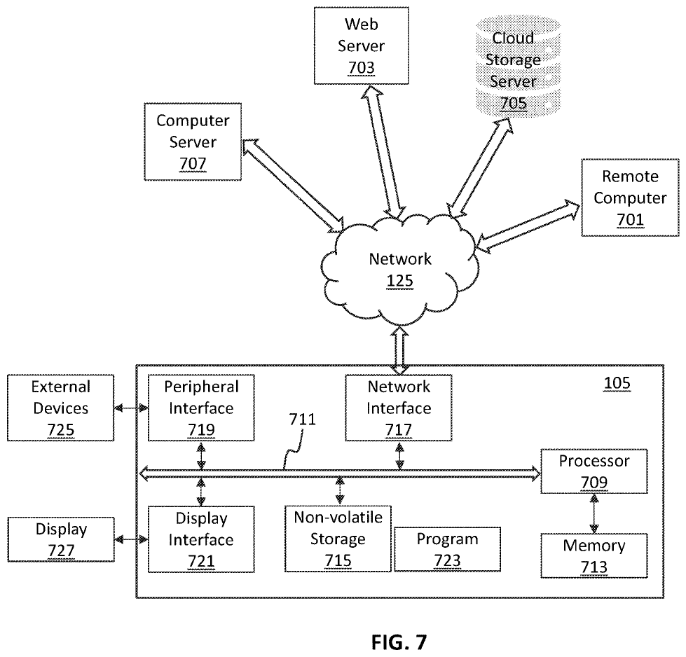

FIG. 7 is a functional block diagram illustrating user device 105 connected to a networked system 700 of one or more networked computers and servers. In an embodiment, the hardware and software environment illustrated in FIG. 7 may provide an exemplary platform for implementation of the software and/or methods according to the present disclosure.

According to some embodiments, a networked system 700 may include, but is not limited to, user device 105, network 125, remote computer 701, web server 703, cloud storage server 705 and computer server 707. Additionally, it should be appreciated that user device 105 can represent one or more of the remote computer 701, web server 703, cloud storage server 705 and computer server 707.

Additional details of user device 105 is shown in FIG. 7. The functional blocks illustrated within user device 105 are provided only to establish exemplary functionality and are not intended to be exhaustive. And while details are not provided for remote computer 701, web server 703, cloud storage server 705 and computer server 707, these other computers and devices may include similar functionality to that shown for user device 105.

User device 105 may be a personal computer (PC), a desktop computer, laptop computer, notebook computer, tablet computer, netbook computer, a personal digital assistant (PDA), a smart phone, or any other programmable electronic device capable of communicating with other devices on network 125.

User device 105 may include processor 709, bus 711, memory 713, non-volatile storage 715, network interface 717, peripheral interface 719 and display interface 721. Each of these functions may be implemented, in some embodiments, as individual electronic subsystems (integrated circuit chip or combination of chips and associated devices), or, in other embodiments, some combination of functions may be implemented on a single chip (sometimes called a system on chip or SoC).

Processor 709 may be one or more single or multi-chip microprocessors, such as those designed and/or manufactured by Intel Corporation, Advanced Micro Devices, Inc. (AMD), Arm Holdings (Arm), Apple Computer, or the like. Examples of microprocessors include Celeron, Pentium, Core i3, Core i5 and Core i7 from Intel Corporation; Opteron, Phenom, Athlon, Turion and Ryzen from AMD; and Cortex-A, Cortex-Rand Cortex-M from Arm.

Bus 711 may be a proprietary or industry standard high-speed parallel or serial peripheral interconnect bus, such as ISA, PCI, PCI Express (PCI-e), AGP, and the like.

Memory 713 and non-volatile storage 715 may be computer-readable storage media. Memory 713 may include any suitable volatile storage devices such as Dynamic Random Access Memory (DRAM) and Static Random Access Memory (SRAM). Non-volatile storage 715 may include one or more of the following: flexible disk, hard disk, solid-state drive (SSD), read-only memory (ROM), erasable programmable read-only memory (EPROM or Flash), compact disc (CD or CD-ROM), digital versatile disk (DVD) and memory card or stick.

Program 723 may be a collection of machine-readable instructions and/or data that is stored in non-volatile storage 715 and is used to create, manage, and control certain software functions that are discussed in detail elsewhere in the present disclosure and illustrated in the drawings. In some embodiments, memory 713 may be considerably faster than non-volatile storage 715. In such embodiments, Program 723 may be transferred from non-volatile storage 715 to memory 713 prior to execution by processor 709.

User device 105 may be capable of communicating and interacting with other computers via network 125 through network interface 717. Network 125 may be, for example, a local area network (LAN), a wide area network (WAN) such as the Internet, or a combination of the two, and may include wired, wireless, or fiber optic connections. In general, network 125 can be any combination of connections and protocols that support communications between two or more computers and related devices.

Peripheral interface 719 may allow for input and output of data with other devices that may be connected locally with user device 105. For example, peripheral interface 719 may provide a connection to external devices 725. External devices 725 may include devices such as a keyboard, a mouse, a keypad, a touch screen, and/or other suitable input devices. External devices 725 may also include portable computer-readable storage media such as, for example, thumb drives, portable optical or magnetic disks, and memory cards. Software and data used to practice embodiments of the present disclosure, for example, Program 723, may be stored on such portable computer-readable storage media. In such embodiments, software may be loaded onto non-volatile storage 715 or, alternatively, directly into memory 713 via peripheral interface 719. Peripheral interface 719 may use an industry standard connection, such as RS-232 or Universal Serial Bus (USB), to connect with external devices 725.

Display interface 721 may connect user device 105 to display 727. Display 727 may be used, in some embodiments, to present a command line or graphical user interface to a user of user device 105. Display interface 721 may connect to display 727 using one or more proprietary or industry standard connections, such as VGA, DVI, DisplayPort and HDMI.

As described above, network interface 717 provides for communications with other computing and storage systems or devices external to user device 105. Software programs and data discussed herein may be downloaded from, for example, remote computer 701, web server 703, cloud storage server 705 and computer server 707 to non-volatile storage 715 through network, interface 717 and network 125. Furthermore, the systems and methods described in this disclosure may be executed by one or more computers connected to user device 105 through network interface 717 and network 125. For example, in some embodiments the systems and methods described in this disclosure may be executed by remote computer 701, computer server 707, or a combination of the interconnected computers on network 125. Mobile applications, subscription website data, datasets and/or databases employed in embodiments of the systems and methods described in this disclosure may be stored and or downloaded from one or more of the remote computer 701, web server 703, cloud storage server 705 and computer server 707.

As an example of a first clinical scenario, after having knee replacement surgery, a patient of 65 years of age is administered a treatment to the proximal quadriceps tendon using various vibration amplitudes between about 0.5 mm and about 3 mm and frequencies between about 80 Hz and about 120 Hz for at least 20 minutes once a day. Following treatment, the patient's recovery from the knee replacement surgery accelerates as compared to a patient who does not receive the treatment. After a short period of time, the treating physician determines that the patient has recovered from the replacement knee surgery and such time to recovery was shorter than that for a patient not receiving the treatment.

As another example of a second clinical scenario, a patient of 45 years of age that is suffering from upper crossed syndrome, is administered a treatment plan to the retractor musculature mid-back and lower trapezius muscles using various vibration amplitudes between about 0.5 mm and about 1.5 mm and frequencies between about 60 Hz and about 120 Hz for 10-15 minutes at least once a day. Following treatment, the patient's recovery from the upper crossed syndrome accelerates as compared to a patient who does not receive the treatment. Upon completion of the treatment plan, the treating physician determines that the patient has improved posture and muscle balance in the shoulders, neck and back. Time to correct the posture and muscle balance was shorter than that for a patient not receiving the treatment.

As still another example of a third clinical scenario, a patient of 25 years of age that is suffering from an acute ankle sprain, is administered a treatment plan to the distal anterior tibialis tendon, the proximal gastrocnemius tendon, and/or the distal Achilles tendon using various vibration amplitudes between about 0.1 mm and about 0.5 mm and frequencies between about 60 Hz and about 120 Hz for up to 60 minutes at least 3 times per week. Following treatment, the patient's recovery from the acute ankle sprain accelerates as compared to a patient who does not receive the treatment. Upon completion of the treatment plan, the treating physician determines that the patient's ankle has improved mobility, reduced swelling, and increased strength. Time to heal the acute ankle sprain was shorter than that for a patient not receiving the treatment.

In yet another example of a fourth clinical scenario, after having hip replacement surgery, a patient of 75 years of age is administered a treatment plan to the gluteal musculature tendon using various vibration amplitudes between about 0.5 mm and about 3.0 mm and frequencies between about 80 Hz and about 120 Hz for at least 60 minutes 2-3 times per week. Following treatment, the patient's recovery from the hip replacement surgery accelerates as compared to a patient who does not receive the treatment. Upon completion of the treatment plan, the treating physician determines that the patient has recovered from the hip replacement surgery and such time to recovery was shorter than that for a patient not receiving the treatment.

In still yet another example of a fifth clinical scenario, a patient of 55 years of age that is suffering from osteoarthritis of the knees, is administered a treatment plan to the proximal quadriceps tendon using various vibration amplitudes between about 0.5 mm and about 3.0 mm and frequencies between about 80 Hz and about 120 Hz for up to 20 minutes once a day. Following treatment, the patient's recovery from the osteoarthritis of the knee accelerates as compared to a patient who does not receive the treatment. Upon completion of the treatment plan, the treating physician determines that the patient's knee has improved mobility, reduced swelling, and increased strength. Time to relieve the symptoms of the osteoarthritis of the knee was shorter than that for a patient not receiving the treatment.

In yet still another example of a sixth clinical scenario, a patient of 45 years of age that is suffering from mechanical low back pain is administered a treatment plan to the gluteal musculature, paraspinals, quadriceps, and/or hamstrings using various vibration amplitudes between about 0.5 mm and about 3.0 mm and frequencies between about 40 Hz and about 120 Hz with an average frequency between about 80 Hz and about 120 Hz for up to 60 minutes 2-3 times per week. Following treatment, the patient's recovery from the mechanical low back pain accelerates as compared to a patient who does not receive the treatment. Upon completion of the treatment plan, the treating physician determines that the patient's back has improved mobility, reduced swelling, and increased strength. Time to relieve the symptoms of the mechanical low back pain was shorter than that for a patient not receiving the treatment.

The control aspects and computational aspects of the present disclosure may be embodied as a system, a method, and/or a computer program product. The computer program product may include a computer readable storage medium on which computer readable program instructions are recorded that may cause one or more processors to carry out aspects of the embodiment.

The computer readable storage medium may be a tangible device that can store instructions for use by an instruction execution device (processor). The computer readable storage medium may be, for example, but is not limited to, an electronic storage device, a magnetic storage device, an optical storage device, an electromagnetic storage device, a semiconductor storage device, or any appropriate combination of these devices. A non-exhaustive list of more specific examples of the computer readable storage medium includes each of the following (and appropriate combinations): flexible disk, hard disk, solid-state drive (SSD), random access memory (RAM), read-only memory (ROM), erasable programmable read-only memory (EPROM or Flash), static random access memory (SRAM), compact disc (CD or CD-ROM), digital versatile disk (DVD) and memory card or stick. A computer readable storage medium, as used in this disclosure, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described in this disclosure can be downloaded to an appropriate computing or processing device from a computer readable storage medium or to an external computer or external storage device via a global network (i.e., the Internet), a local area network, a wide area network and/or a wireless network. The network may include copper transmission wires, optical communication fibers, wireless transmission, routers, firewalls, switches, gateway computers and/or edge servers. A network adapter card or network interface in each computing or processing device may receive computer readable program instructions from the network and forward the computer readable program instructions for storage in a computer readable storage medium within the computing or processing device.

Computer readable program instructions for carrying out operations of the present disclosure may include machine language instructions and/or microcode, which may be compiled or interpreted from source code written in any combination of one or more programming languages, including assembly language, Basic, Fortran, Java, Python, R, C, C++, C#, or similar programming languages. The computer readable program instructions may execute entirely on a user's personal computer, notebook computer, tablet, or smartphone, entirely on a remote computer or computer server, or any combination of these computing devices. The remote computer or computer server may be connected to the user's device or devices through a computer network, including a local area network or a wide area network, or a global network (i.e., the Internet). In some embodiments, electronic circuitry including, for example, programmable logic circuitry, field-programmable gate arrays (FPGA), or programmable logic arrays (PLA) may execute the computer readable program instructions by using information from the computer readable program instructions to configure or customize the electronic circuitry, in order to perform aspects of the present disclosure.

According to some embodiments, a vibration therapy device, includes: a vibration element; and a control unit including a processor configured to determine control parameters for the vibration element based on a treatment plan for a target site of a patient's body; and control an effective frequency and an effective amplitude of a therapeutic vibration of the vibration element, during operation, based on the control parameters, wherein the effective frequency is in a therapeutic range of frequencies based on the treatment plan and the effective amplitude is in a therapeutic range of amplitudes based on the target site of a patient's body. In some embodiments, the vibration therapy device further includes an attachment plate that secures the vibration element to an adjustable strap or clothing worn by the patient. In some embodiments, the attachment plate secures the vibration element to the adjustable strap or clothing by using a magnetic force between the attachment plate and the vibration element. In some embodiments, the treatment plan is for increasing blood flow and the therapeutic range of frequencies is an inclusive range between 80 Hz and 100 Hz. In some embodiments, the treatment plan is for relieving muscle spasms and the therapeutic range of frequencies is an inclusive range between 60 Hz and 120 Hz. In some embodiments, the treatment plan is for rehabilitation and the therapeutic range of frequencies is an inclusive range between 80 Hz and 120 Hz.

In other aspects of this embodiment, a frequency is set to at least 20 Hz, at least 25 Hz, at least 30 Hz, at least 35 Hz, at least 40 Hz, at least 45 Hz at least 50 Hz, at least 55 Hz, at least 60 Hz, at least 65 Hz, at least 70 Hz, at least 75 Hz, at least 80 Hz, at least 85 Hz, at least 90 Hz, at least 95 Hz, at least 100 Hz, at least 105 Hz, at least 110 Hz, at least 115 Hz, at least 120 Hz, at least 125 Hz, at least 130 Hz, at least 135 Hz, at least 140 Hz, or more. In other aspects of this embodiment, a frequency is set to about 50 Hz, about 55 Hz, about 60 Hz, about 65 Hz, about 70 Hz, about 75 Hz, about 80 Hz, about 85 Hz, about 90 Hz, about 95 Hz, about 100 Hz, about 105 Hz, about 110 Hz, about 115 Hz, about 120 Hz, about 125 Hz, about 130 Hz, about 135 Hz, about 140 Hz, or more. In other aspects of this embodiment, a frequency is set to no more than 50 Hz, no more than 55 Hz, no more than 60 Hz, no more than 65, Hz no more than 70 Hz, no more than 75 Hz, no more than 80 Hz, no more than 85 Hz, no more than 90 Hz, no more than 95 Hz, no more than 100 Hz, no more than 105 Hz, no more than 110 Hz, no more than 115 Hz, no more than 120 Hz, no more than 125 Hz, no more than 130 Hz, no more than 135 Hz, no more than 140 Hz or more.

In some embodiments, the processor is further configured to control an effective time of the therapeutic vibration of the vibration element during operation, and wherein the effective time is a therapeutic range of times based on the treatment plan for the target site of the patient's body. According to some embodiments, the therapeutic range of times is an inclusive range between 5 minutes and 30 minutes. In some embodiments, the processor is further configured to determine a target site of the patient's body for placement of the vibration therapy device based on the vibration treatment plan. In some embodiments, the processor is further configured to indicate proper placement of the vibration therapy device at the determined target site.

In other aspects of this embodiment, the therapeutic range of time for treatment is 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours or more hours.

In other aspects of this embodiment, the therapeutic range of time for treatment is at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes, at least 19 minutes, at least 20 minutes, at least 21 minutes, at least 22 minutes, at least 23 minutes, at least 24 minutes, at least 25 minutes, at least 26 minutes, at least 27 minutes, at least 28 minutes, at least 29 minutes, at least 30 minutes, 3 at least 5 minutes, at least 40 minutes, at least 45 minutes, at least 50 minutes, at least 55 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours or more hours.

In other aspects of this embodiment, the therapeutic range of time for treatment is no more than 1 minute, no more than 2 minutes, no more than 3 minutes, no more than 4 minutes, no more than 5 minutes, no more than 6 minutes, no more than 7 minutes, no more than 8 minutes, no more than 9 minutes, no more than 10 minutes, no more than 11 minutes, no more than 12 minutes, no more than 13 minutes, no more than 14 minutes, no more than 15 minutes, no more than 16 minutes, no more than 17 minutes, no more than 18 minutes, no more than 19 minutes, no more than 20 minutes, no more than 21 minutes, no more than 22 minutes, no more than 23 minutes, no more than 24 minutes, no more than 25 minutes, no more than 26 minutes, no more than 27 minutes, no more than 28 minutes, no more than 29 minutes, no more than 30 minutes, 3 no more than 5 minutes, no more than 40 minutes, no more than 45 minutes, no more than 50 minutes, no more than 55 minutes, no more than 1 hour, no more than 2 hours, no more than 3 hours, no more than 4 hours, no more than 5 hours, no more than 6 hours, no more than 7 hours, no more than 8 hours, no more than 9 hours, no more than 10 hours, no more than 11 hours, no more than 12 hours or more hours.

In other aspects of this embodiment, the therapeutic range of time for treatment is about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, 3 about 5 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 11 hours, about 12 hours or more hours.

In accordance with some embodiments, a vibration therapy system includes: a first wearable vibration device and a user device including a device processor configured to determine first control parameters for controlling the first wearable vibration device based on a treatment plan for a first target site of a patient's body; and a transceiver for transmitting the first control parameters to the first wearable vibration device, wherein a first effective frequency and a first effective amplitude of a first therapeutic vibration of the first wearable vibration device, during operation, are generated based on the first control parameters, wherein the first effective frequency is in a first therapeutic range of frequencies based on the treatment plan and the first effective amplitude is in a first therapeutic range of amplitudes based on the first target site of a patient's body. In some embodiments, the treatment plan is for increasing blood flow and the first therapeutic range of frequencies is an inclusive range between 80 Hz and 100 Hz. In some embodiments, the treatment plan is for relieving muscle spasms and the first therapeutic range of frequencies is an inclusive range between 60 Hz and 120 Hz. In some embodiments, the treatment plan is for rehabilitation and the first therapeutic range of frequencies is an inclusive range between 40 Hz and 80 Hz. In some embodiments, the treatment plan is for rehabilitation and the first therapeutic range of frequencies is an inclusive range between 80 Hz and 120 Hz. In some embodiments, the vibration therapy system further includes a second wearable vibration device, wherein the device processor is further configured to determine second control parameters for controlling the second wearable vibration device based on the treatment plan for a second target site of the patient's body; the transceiver is further configured to transmit the second control parameters to the second wearable vibration device; and wherein a second effective frequency and a second effective amplitude of a second therapeutic vibration of the second wearable vibration device, during operation, are generated based on the second control parameters, wherein the second effective frequency is in a second therapeutic range of frequencies based on the treatment plan and the second effective amplitude is in a second therapeutic range of amplitudes based on the second target site of a patient's body. In some embodiments, the second effective amplitude of the second therapeutic vibration generated by the second wearable vibration device is different from the effective amplitude of the therapeutic vibration generated by the wearable vibration device. In some embodiments, the second target site of the patient's body is at a location of an antagonistic pairing muscle to a first muscle undergoing treatment at the first target site of the patient's body. In some embodiments, the vibration therapy system further includes a camera configured to capture video of the user placing the first wearable vibration device at the first target site; and wherein the device processor is further configured to determine appropriate positioning of the first wearable vibration device using the video captured by the camera.

Aspects of the present specification disclose, in part, treating an individual suffering from a clinical syndrome or disease. As used herein, the term “treating,” refers to reducing or eliminating in an individual a clinical syndrome or disease; or delaying or preventing in an individual the onset of a clinical syndrome or disease. For example, the term “treating” can mean reducing a symptom of a condition characterized by a syndrome or disease, including a reduction or elimination of pain or expediting the growth of new tissue, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with the disclosed syndromes and diseases are well known and can be determined by a person of ordinary skill in the art by taking into account factors, including, without limitation, the location of the syndrome or disease in the body, including pain, the location of the pain and the genesis of the pain. Those of skill in the art will know the appropriate symptoms or indicators associated with a specific type of syndrome or diseases, including pain, and will know how to determine if an individual is a candidate for treatment as disclosed herein.

In aspects of this embodiment, a variable vibration frequency, amplitude and duration used as part of vibration therapy reduces a symptom associated with injury recovery, sore muscles, muscle spasms, release soft tissues and or blood flow by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In aspects of this embodiment, a variable vibration frequency, amplitude and duration used as part of vibration therapy reduces a symptom associated with injury recovery, sore muscles, muscle spasms, release soft tissues and or blood flow by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%.

In aspects of this embodiment, a variable vibration frequency, amplitude and duration used as part of vibration therapy reduces a symptom associated with injury recovery, sore muscles, muscle spasms, release soft tissues and or blood flow by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In aspects of this embodiment, a variable vibration frequency, amplitude and duration used as part of vibration therapy reduces pain, recovery time for a healing tissue, improved muscle strength, and or circulation by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In aspects of this embodiment, a variable vibration frequency, amplitude and duration used as part of vibration therapy reduces pain, recovery time for a healing tissue, improved muscle strength, and or circulation by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%.

In aspects of this embodiment, a variable vibration frequency, amplitude and duration used as part of vibration therapy reduces pain, recovery time for a healing tissue, improved muscle strength, and or circulation by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In aspects of this embodiment, a variable vibration frequency, amplitude and duration used as part of vibration therapy reduces the severity of back pain, lower back pain, upper crossed syndrome, post-surgical hip and/or knee surgery recovery (including replacement surgery), tendon damage, ligament damage, damage to the anterior tibialis tendon, damage to the proximal gastrocnemius tendon, damage to the Achilles tendon, damage to the distal Achilles tendon, a sprain, an ankle sprain, tennis elbow and other tissue, ligament or tendon damage by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In aspects of this embodiment, a variable vibration frequency, amplitude and duration used as part of vibration therapy reduces the severity of back pain, lower back pain, upper crossed syndrome, post-surgical hip and/or knee surgery recovery (including replacement surgery), tendon damage, ligament damage, damage to the anterior tibialis tendon, damage to the proximal gastrocnemius tendon, damage to the Achilles tendon, damage to the distal Achilles tendon, a sprain, an ankle sprain, tennis elbow and other tissue, ligament or tendon damage by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%.

In aspects of this embodiment, a variable vibration frequency, amplitude and duration used as part of vibration therapy reduces the severity of back pain, lower back pain, upper crossed syndrome, post-surgical hip and/or knee surgery recovery (including replacement surgery), tendon damage, ligament damage, damage to the anterior tibialis tendon, damage to the proximal gastrocnemius tendon, damage to the Achilles tendon, damage to the distal Achilles tendon, a sprain, an ankle sprain, tennis elbow and other tissue, ligament or tendon damage by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In aspects of this embodiment, a variable vibration frequency, amplitude and duration used as part of vibration therapy reduces the time to heal ligament sprains, muscle strains, tendonitis, joint inflammation, plantar fasciitis, metatarsalgia, facet irritation, impingement syndrome, bursitis, rheumatoid arthritis, osteoarthritis and scar tissue adhesion by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In aspects of this embodiment, a variable vibration frequency, amplitude and duration used as part of vibration therapy reduces the time to heal ligament sprains, muscle strains, tendonitis, joint inflammation, plantar fasciitis, metatarsalgia, facet irritation, impingement syndrome, bursitis, rheumatoid arthritis, osteoarthritis and scar tissue adhesion by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%.

In aspects of this embodiment, a variable vibration frequency, amplitude and duration used as part of vibration therapy reduces the time to heal ligament sprains, muscle strains, tendonitis, joint inflammation, plantar fasciitis, metatarsalgia, facet irritation, impingement syndrome, bursitis, rheumatoid arthritis, osteoarthritis and scar tissue adhesion by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

Treatment can be conducted once, or multiple times and the number of treatments can be readily determined by one skilled in the art. For instance, treatment of, in an embodiment, a pain or tissue healing, may comprise a one-time treatment. Alternatively, treatment of, in an embodiment, a pain or tissue healing, may comprise multiple treatments at an effective frequency and amplitude carried out over a range of time periods, such as, e.g., once daily, twice daily, trice daily, once every few days, or once weekly. The timing of treatment can vary from individual to individual, depending upon such factors as the severity of an individual's symptoms. For example, an effective treatment disclosed herein can be administered to an individual once daily for an indefinite period of time, or until the individual no longer requires therapy. A person of ordinary skill in the art will recognize that the condition of the individual can be monitored throughout the course of treatment and that the effective amount of a treatment disclosed herein for, in an embodiment, pain or tissue healing, that is administered can be adjusted accordingly.

In an embodiment, the period of treatment of a patient is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of treatment during which treatment of a patient is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In accordance with some aspects of the disclosed subject matter, a method of using a wearable vibration therapy device includes determining control parameters for controlling the wearable vibration device based on a treatment plan for a target site of a patient's body; providing the control parameters to the wearable vibration therapy device; generating an effective frequency and an effective amplitude of a therapeutic vibration via the wearable vibration device, based on the control parameters, wherein the effective frequency is in a therapeutic range of frequencies based on the treatment plan and the effective amplitude is in a therapeutic range of amplitudes based on the target site of a patient's body. In some embodiments, the method further includes determining that the wearable vibration device is positioned at the target site of the patient's body; and generating an indication of appropriate placement of the wearable vibration device.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context. \Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical ranges or value, however, inherently contains certain errors necessarily resulting from the standard deviation found in their respective testing measurements. Recitation of numerical ranges of values herein is merely intended to serve as a shorthand method of referring individually to each separate numerical value falling within the ranges. Unless otherwise indicated herein, each individual value of a numerical ranges is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of’ excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A vibration therapy device, comprising: a vibration element; and
a control unit comprising a processor configured to determine control parameters for the vibration element based on a treatment plan for a target site of a patient's body; and
control an effective frequency and an effective amplitude of a therapeutic vibration of the vibration element, during operation, based on the control parameters, wherein the effective frequency is in a therapeutic range of frequencies based on the treatment plan and the effective amplitude is in a therapeutic range of amplitudes based on the target site of a patient's body.

2. The vibration therapy device of claim 1 further comprises an attachment plate that secures the vibration element to an adjustable strap or clothing worn by the patient.

3. The vibration therapy device of claim 2, wherein the attachment plate secures the vibration element to the adjustable strap or clothing by using a magnetic force between the attachment plate and the vibration element.

4. The vibration therapy device of claim 1, wherein the treatment plan is for increasing blood flow, the therapeutic range of frequencies is an inclusive range between 40 Hz and 100 Hz.

5. (canceled)

6. The vibration therapy device of claim 1, wherein the treatment plan is for rehabilitation, the therapeutic range of frequencies is an inclusive range between 40 Hz and 120 Hz with an average frequency of 80 Hz to 100 Hz.

7. The vibration therapy device of claim 1, wherein the processor is further configured to control an effective time of the therapeutic vibration of the vibration element during operation, and wherein the effective time is a therapeutic range of times based on the treatment plan for the target site of the patient's body.

8. The vibration therapy device of claim 7, wherein the therapeutic range of times is an inclusive range between 5 minutes and 60 minutes.

9. (canceled)

10. (canceled)

11. A vibration therapy system, comprising:
a first wearable vibration device; and a user device comprising:
a device processor configured to determine first control parameters for controlling the first wearable vibration device based on a treatment plan for a first target site of a patient's body; and
a transceiver for transmitting the first control parameters to the first wearable vibration device, wherein a first effective frequency and a first effective amplitude of a first therapeutic vibration of the first wearable vibration device, during operation, are generated based on the first control parameters, wherein the first effective frequency is in a first therapeutic range of frequencies based on the treatment plan and the first effective amplitude is in a first therapeutic range of amplitudes based on the first target site of a patient's body; and
a second wearable vibration device,
wherein the device processor is further configured to determine second control parameters for controlling the second wearable vibration device based on the treatment plan for a second target site of the patient's body;
the transceiver is further configured to transmit the second control parameters to the second wearable vibration device; and
wherein a second effective frequency and a second effective amplitude of a second therapeutic vibration of the second wearable vibration device, during operation, are generated based on the second control parameters,
wherein the second effective frequency is in a second therapeutic range of frequencies based on the treatment plan and the second effective amplitude is in a second therapeutic range of amplitudes based on the second target site of a patient's body,
wherein the second effective amplitude of the second therapeutic vibration generated by the second wearable vibration device is different from the effective amplitude of the therapeutic vibration generated by the wearable vibration device; and
wherein the second target site of the patient's body is at a location of an antagonistic pairing muscle to a first muscle undergoing treatment at the first target site of the patient's body.

12. The vibration therapy system of claim 11, wherein the treatment plan is for increasing blood flow, the first therapeutic range of frequencies is an inclusive range between 40 Hz and 80 Hz.

13. The vibration therapy system of claim 1, wherein the treatment plan is for relieving muscle spasms, the first therapeutic range of frequencies is an inclusive range between 60 Hz and 120 Hz.

14. The vibration therapy system of claim 11, wherein the treatment plan is for neuromuscular rehabilitation, the first therapeutic range of frequencies is an inclusive range between 40 Hz and 120 Hz with an average frequency of 80 Hz to 100 Hz.

15. (canceled)

16. (canceled)

17. (canceled)

18. The vibration therapy system of claim 11 further comprising:
a camera configured to capture video of the user placing the first wearable vibration device at the first target site; and
wherein the device processor is further configured to determine appropriate positioning of the first wearable vibration device using the video captured by the camera.

19. A method of using a wearable vibration therapy device, the method comprising: determining control parameters for controlling the wearable vibration device based on a
treatment plan for a target site of a patient's body;
providing the control parameters to the wearable vibration therapy device;
generating an effective frequency and an effective amplitude of a therapeutic vibration via the wearable vibration device, based on the control parameters, wherein the effective frequency is in a therapeutic range of frequencies based on the treatment plan and the effective amplitude is in a therapeutic range of amplitudes based on the target site of a patient's body.

20. The method of claim 19 further comprising:
determining that the wearable vibration device is positioned at the target site of the patient's body; and
generating an indication of appropriate placement of the wearable vibration device.

21. The method of claim 19, wherein the treatment plan treats mechanical low back pain, and the target site of the patient's body is one or more of the gluteal musculature, the paraspinals, the quadriceps and/or the hamstrings.

22. The method of claim 19, wherein the treatment plan treats osteoarthritis of the knee, and the target site of the patient's body is one or more of the proximal quadriceps tendon.

23. The method of claim 19, wherein the treatment plan treats upper crossed syndrome, and the target site of the patient's body is one or more of the retractor musculature mid-back and/or the lower trapezius.

24. The method of claim 19, wherein the treatment plan treats a post-surgical hip replacement, and the target site of the patient's body is one or more of the gluteal musculature tendons.

25. The method of claim 19, wherein the treatment plan treats a post-surgical knee replacement, and the target site of the patient's body is one or more of the proximal quadriceps tendon.

26. The method of claim 19, wherein the treatment plan treats an acute ankle sprain, and the target site of the patient's body is one or more of the distal anterior tibialis tendon, proximal gastrocnemius tendon, and/or the distal Achilles tendon.
